(19) United States
(12) Patent Application Publication
(10) Pub. No.: US 2011/0256117 A1
(43) Pub. Date: Oct. 20, 2011

(54) MANUFACTURING AND USE OF COMPOSITE SCAFFOLDS

(75) Inventors: Hanry Yu, Singapore (SG); Yuet Mei Khong, Singapore (SG); Feng Wen, Singapore (SG)

(73) Assignee: Agency for Science, Technology and Research

(21) Appl. No.: 13/060,111

(22) PCT Filed: Aug. 24, 2009
(86) PCT No.: PCT/SG09/00296

§ 371 Date: Jul. 11, 2011

Related U.S. Application Data
(60) Provisional application No. 61/136,276, filed on Aug. 22, 2008.

(51) Int. Cl. A61K 38/43; C12N 1/00; A61K 31/7088; A61K 38/02; A61K 47/42; A61P 31/12; A61P 7/02; A61P 7/00; A61P 29/00; A61P 37/06; A61P 31/00; C12N 5/071; A61P 35/00

(52) U.S. Cl. 424/94.1; 435/395; 435/243; 514/44 A; 514/44 R; 514/1.1; 514/772.1

(57) ABSTRACT

The present invention refers to a method of manufacturing a composite scaffold including the surface grafting of the scaffold using gamma-irradiation as well as gamma-irradiation of a polymer contacted with the surface of the scaffold. The present invention also directed to the use of the scaffold for tissue engineering and other applications. The present invention is further directed to a method of culturing cells in a composite scaffold of the present invention and to a three-dimensional porous composite scaffold.

### CROSS-REFERENCE TO RELATED APPLICATIONS

This application claims the benefit of priority of U.S. provisional application No. 61/136,276, filed Aug. 22, 2008, the contents of it being hereby incorporated by reference in its entirety for all purposes.

### FIELD OF THE INVENTION

The present invention refers to the field of chemistry, in particular the chemistry regarding the manufacture of scaffolds which can be used for tissue engineering, drug delivery and cell seeding purposes.

### BACKGROUND OF THE INVENTION

Biodegradable materials are increasingly used as scaffolds in tissue engineering or as drug delivery carriers. These scaffolds must meet criteria of controllable mechanical properties and biodegradability, as well as biocompatibility. Activation of surfaces on these scaffolds is also critical to ensure optimal cellular response. Another essential scaffold characteristic is uniformity, which includes uniform distribution of active ligands on scaffold surfaces, and mechanical support and porosity to buttress cells. Precision control of these properties involve integration of various materials of different physical and biochemical properties to create materials with a melange of desirable characteristics. However, more-often-than-not, researchers encounter difficulties in balancing economic trade-offs as well as industrial scalability in order to achieve an ideal scaffold for specific applications.

There are for example, various methods to induce surface modification on the surface of hydrophobic materials. Some of these techniques include dehydrothermal crosslinking, chemical crosslinking and irradiation using ionizing agents such as UV light. These techniques face several critical disadvantages such as (i) non-uniform distribution of activated surfaces and crosslinking, (ii) difficulty in removing chemicals that had been used resulting in cytotoxicity in subsequent cell applications.

Thus, a need exists to provide further materials for the manufacture of scaffolds which meet at least some of the above needs.

### SUMMARY OF THE INVENTION

In a first aspect, the present invention refers to a method of manufacturing a composite scaffold. The method comprises the steps (in one embodiment in the following order) of:

irradiating a three dimensional porous scaffold with gamma-rays;

contacting the scaffold in a solution comprising an unsaturated carboxylic acid for introducing carboxyl groups at the surface of the scaffold;

activating the carboxyl groups to obtain activated carboxyl groups;

contacting the scaffold comprising the activated carboxyl groups with a first polymer solution in a first contacting step to allow reaction of the activated carboxyl groups with the polymer in the polymer solution;

contacting the scaffold in a second contacting step with a second polymer solution; and

irradiating the scaffold which is in contact with the second polymer solution with gamma-rays to obtain the composite scaffold.

In a further aspect, the present invention refers to a method of manufacturing a composite scaffold seeded with cells. This method comprises providing a composite scaffold as referred to in any one of claims 1 to 34; and seeding cells into the composite scaffold.

In still another aspect, the present invention refers to a scaffold obtained or obtainable by a method as described herein.

In still a further aspect, the present invention refers to the use of a composite scaffold referred to herein for tissue engineering and/or and drug delivery or cell culturing.

In still a further aspect, the present invention refers to a porous three-dimensional composite scaffold having a compressive modulus of between about 5 to 50 MPa and having a pore size of between about 20 to 200 μm.

### BRIEF DESCRIPTION OF THE DRAWINGS

The invention will be better understood with reference to the detailed description when considered in conjunction with the non-limiting examples and the accompanying drawings, in which:

FIG. 1 shows a general overview of an embodiment of the method of the present invention.

FIG. 2 illustrates an embodiment of the invention in which the manufacture of a composite scaffold is illustrated. The composite scaffold manufactured as outlined in the experimental section is a PCL-collagen scaffold.

FIG. 3 shows the spatial distributions of: FIG. 3(A) carboxyl groups and FIG. 3(B) amine groups through the scaffolds (PCLA or PCLAC) determined by Toluidine blue O and Acid Orange assays. All data were normalized against the concentration on the top surface of each respective scaffold. The results showed concentration gradients for both carboxyl groups and amine groups on the surfaces of PCLA and PCLAC scaffolds whereby surface grafting was induced by plasma treatment (PCLAp and PCLACp), but not for scaffolds whereby surface grafting was induced by gamma irradiation (PCLAg and PCLACg).

FIG. 4 shows the results of the characterization of the PCL-collagen scaffolds by evaluating FIG. 4(A). Compression modulus of the scaffolds. FIG. 4(B) Pore size of the scaffolds. It was found that the collagen sponge without the PCL frame has very low compression modulus and the PCL scaffold has 4 times higher compression modulus than collagen sponge. The mechanical strength of the scaffold increased significantly in comparison to PCL scaffold alone after cross-linking collagen to the PCL scaffold. Furthermore, as shown in FIG. 4(B) about 70% of the pore volume has a pore size of between 100 μm to 180 μm and the rest of the 30% of the pore volume has a pore size of less than 100 μm.

FIG. 5 shows scanning electron microscope images of PCL-collagen scaffolds taken from FIG. 5(A). Middle of scaffold, FIG. 5(B) Top of the scaffold. Black arrows show the PCL framework whereas white arrows show the collagen plates. The scanning electron microscope images show the PCL framework, covered with cross-linked collagen plates, thus, significantly increases the area for cells attachment. The scaffolds are highly porous and have uniform distribution of collagen on the top and middle. This is concurrent with the data obtained using the porosimeter. Scale bars 100 μm.

FIG. 6 shows SEM micrographs of the morphologies of hepatocytes in: FIG. 6(A) collagen sponge; FIG. 6(B) PCL scaffold; and FIG. 6(C) PCL-collagen scaffolds after 24 h of cell seeding. As can be seen from the separate images, few hepatocytes were attached on the surface of PCL scaffold (FIG. 6(B)) and were present in singles or in pairs. Some hepatocytes also attached on the surface of collagen strips in collagen sponge in singles or in pairs (FIG. 6(A)). In contrast, many hepatocytes attached on the surface of collagen strips or PCL microfibers in PCL-collagen scaffold (FIG. 6(C)) as cell aggregates. Scale bars 100 μm.

FIG. 7 shows the number of hepatocytes attached in different scaffolds at 24 h after cell seeding (normalised by the number of cells seeded) (n=3, mean±SD).

FIG. 8 shows reduction of AlamarBlue™ by hepatocytes cultured in different scaffolds at 24 h after cell seeding (n=3, mean±SD). The results illustrated in FIG. 8 indicate that the reduction of AlamarBlue by hepatocytes cultured in the PCL-collagen scaffold was much higher than in collagen sponge or PCL scaffold.

FIG. 9 shows an image of a PCL scaffold hold in place with a forceps. Scale bar 1000 μm. Scaffolds having this or similar structures can be manufactured using any material referred to herein. FIG. 9 illustrates the network of interconnected pores in a scaffold which is formed by the polymeric backbone structure.

### DETAILED DESCRIPTION OF THE PRESENT INVENTION

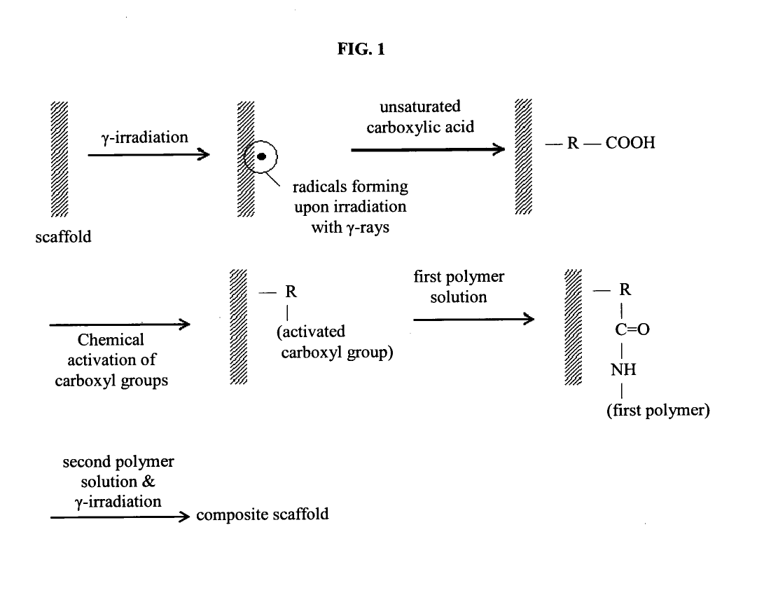

In a first aspect, the present invention refers to a method of manufacturing a composite scaffold. The method comprises in a first step irradiation of a three dimensional porous scaffold with gamma-rays (γ-rays). Gamma irradiation of the scaffold forms free radicals on the surface of the scaffold. In a further step the method comprises contacting of the gamma irradiated scaffold in a solution comprising an unsaturated carboxylic acid for introducing carboxyl groups on the surface of the gamma-irradiated scaffold. The unsaturated carboxylic acid reacts with the radicals which have been formed by gamma-irradiation at the surface of the scaffold. In a further step the method comprises the activation of the carboxyl groups introduced on the surface of the scaffold using the unsaturated carboxylic acid for obtaining activated carboxyl groups which can react in a next step with a polymer comprised in a first polymer solution which is brought into contact with the grafted scaffold, i.e. the scaffold comprising the carboxyl groups or activated carboxyl groups on its surface. In a further step the method comprises contacting the scaffold in a second contacting step with a second polymer solution. While being in contact with the second polymer solution, the scaffold is irradiated with gamma-rays which leads to cross-linking of the polymer of the second polymer solution in the scaffold and thus leads to the final formation of the composite scaffold. The above method is illustrated in FIG. 1.

Using this method allows to fabricate a composite material that can be precisely tuned to possess characteristics of bioactive functional surface, optimal mechanical properties and controllable biodegradability. As described above, the method encompasses surface modification of a hydrophobic scaffold with collagen via gamma-irradiation, followed by collagen crosslinking with the same method. The gamma-irradiation technique used in this embodiment induces primarily crosslinking in the wet state in comparison to chain-scission in the dry state. A mechanically stable scaffold which can be fabricated, for example by the highly reproducible and computer-controlled fused deposition modelling technique and which is a commercialized product that has been approved by the United State Food and Drug Administration (FDA), such as poly-ε-caprolactone, is used as the backbone and a further polymer, such as collagen, is used to fill up the scaffold to create more surfaces and interconnected micropores for cellular support and cell-biomaterial interaction. Cellular compatibility of such a composite scaffold has been demonstrated by primary rat hepatocytes culture (see the experimental section). An important advantage of the current technique is the ease of fabrication using gamma-irradiation as a low-cost modification technique to create composite scaffolds with uniform ligands distribution, porosity and scalable for commercial applications.

The use of gamma-irradiation in the method referred to herein enables uniform exposure of the scaffold as well as a second polymer solution to gamma-irradiation, thus, facilitating the entire large scaffold to undergo the same free-radical and cross-linking reaction. Utilization of gamma irradiation negates the need to use chemical initiators and subsequent processes to cleanse the scaffolds. Scaffolds generated using the method referred to herein provide optimal conditions to cells adhesion as well as functions. At the same time, gamma-irradiation is a commonly used technique for sterilization of biomedical devices. Hence, it is possible to integrate this technique of fabrication with the sterilization process in an all-in-one simple step, further enhancing its appeal for commercial applications.

Furthermore, the dosage of the irradiation can be precisely controlled to modulate the mechanical properties, porosity and also biodegradability as illustrated in the experimental section. Also, gamma-rays are highly penetrative electromagnetic radiation, therefore, scaffolds of any size can be modified and processed using this method. This characteristic is particularly attractive for commercialization because it can be effortlessly scaled up at relatively low costs.

In addition, the current method is applicable to many types of scaffold materials and second polymer solution combinations, where the scaffold provides precision mechanical framework and the second polymer solution can increase surface area, e.g., for cell-biomaterial interaction.

A “composite scaffold” (can also be called “hybrid scaffold”) is comprised of a combination of two or more materials, differing in form or composition on a macroscale. In the composite scaffold of the present invention those materials are the scaffold, the polymer in the first polymer solution and the polymer in the second polymer solution. The constituents retain their identities, that is, they do not dissolve or merge completely into one another although they act in concert. Normally, the scaffold material and the polymers of the polymer solutions can be physically identified and exhibit an interface between one another.

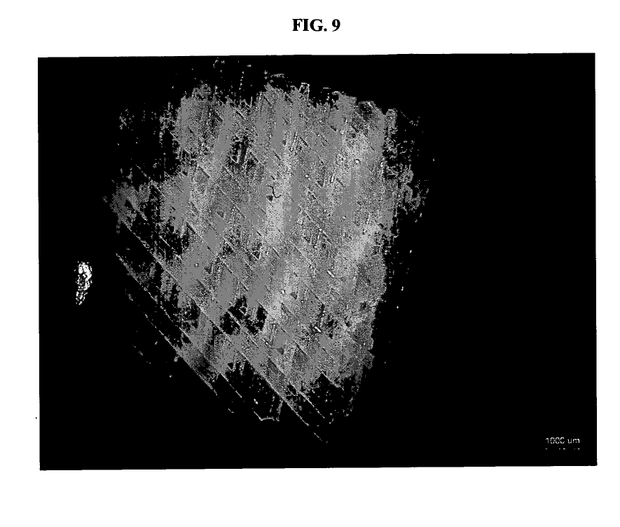

A “scaffold” as used in the method referred to herein is an engineered pre-existing structure having a network of interconnected pores. The scaffold is porous and the scaffold or framework structure provides the backbone for the construction of the porous composite scaffold. In one embodiment, the scaffold provides a regular and uniform porous structure, which means that the structural elements which the scaffold is composed of repeat itself. Such scaffolds are mostly manufactured synthetically in contrast to scaffolds obtained from tissue and can be manufactured using common techniques, such as leaching method, phase separation method, electrospinning method, printing and prototyping method, to name only a few. An exemplary picture of such a scaffold is shown in FIG. 9 which shows a PCL scaffold.

In another embodiment, the backbone structure of the scaffold is irregular. Such irregular scaffold structures are obtained when for example decelluralizing an existing scaffold structure obtained from existing tissue structure. The scaffold is comprised of a network of interconnected pores. The size of the pores in such a network is variable and can range between several hundred nanometers or micrometers to millimeters. In one embodiment, the pore dimensions are in the range of between about 500 nm to about 400 μm. In another embodiment, the pore size can be between about 10 μm to about 200 μm.

Scaffolds play for example a critical role in tissue engineering. The function of scaffolds is to direct, for example, the growth of cells either seeded within the porous structure of the scaffold or migrating from surrounding tissue into the scaffold. The majority of mammalian cell types are anchorage-dependent, meaning they will die if an adhesion substrate is not provided. Porous scaffold matrices as disclosed herein can be used to achieve cell delivery with high loading and efficiency to specific sites. Therefore, the scaffold should provide a suitable substrate for cell attachment, cell proliferation, differentiated function, and cell migration.

The prerequisite physicochemical properties of scaffolds are many: to support and deliver cells; induce, differentiate, and channel tissue growth; target cell-adhesion substrates; stimulate cellular response; provide a wound-healing barrier; be biocompatible and/or biodegradable; possess relatively easy processability and malleability into desired shapes; be highly porous with a large surface/volume ratio; possess mechanical strength and dimensional stability; and have sterilisability, among others.

For example, the polymer in the second polymer solution which is cross-linked into the pre-existing scaffold provides an ideal substrate for cell attachment and survival. This is of particular importance for anchorage-dependent cells which constitute most of the cells in the human body. However, the composite scaffold referred to herein can also be used for non-anchorage-dependent cells. The method referred to herein allows the versatility to control the mechanical stability and biodegradability and also achieve uniform distribution of bioactive ligands and porosity. It is also possible to cross-link further chemical compounds, such as pharmaceuticals or growth-factors or specific antibodies into the composite scaffold structure as explained in more detail further below.

Generally, a three-dimensional porous scaffold is a scaffold which is fabricated from a hydrophobic polymer. In one embodiment, the scaffold is manufactured of a hydrophobic non-biodegradable (permanent) and biodegradable material. Biodegradability is defined as the ability of a material to enzymatically or non-enzymatically degrade and disappear form the original body site, i.e. the site of the body the scaffold has been implanted.

Examples for non-biodegradable polymers which the scaffold can be made of include, but are not limited for polystyrene, polyvinylalcohol (PVA), polyhydroxyethyl-methacrylate (pHEMA) or poly(N-isopropylacrylamide) (PNIPAAm). All polymers referred to herein and in particular non-biodegradable polymers can be used for example for prosthetic devices. For example polystyrene scaffolds (Prolene) are used for hernia repair.

Biodegradable polymeric materials of which scaffolds can be made of can be a synthetic polymeric material or a natural polymeric material or blends thereof. A synthetic polymeric material differs in general from a natural polymeric material in the way they are degraded by an organism. Synthetic polymers are degraded by hydrolysis although polyamino acids show degradation via enzymes, while natural polymers are degraded enzymatically.

In one embodiment, examples of synthetic biodegradable polymeric materials include, but are not limited to poly(α-hydroxy esters), such as polyglycolide (PGA), polylactide (PLA) and its copolymer poly(lactide-co-glycolide) (PLGA), polyphosphazene, polyanhydride, polypropylene fumarate), polycyanoacrylate, poly-ε-caprolactone (PCL), poly-dioxanone (PDO) or biodegradable polyurethanes.

In another embodiment, synthetic biodegradable polymeric materials include, but are not limited to polylactides, polyglycolides, lactide/glycolide copolymers, polycaprolactone, poly(p-dioxane), poly(β-malic acid), poly(anhydrides), poly(ortho esters), polycarbonates, poly(phosphazenes), poly(amino acids), poly(phosphoric ester-urethanes), poly(cyanoacrylates), polyethylene, polyurethane, poly(butyl acrylate), poly(methyl methacrylate), poly(ethylene terephthalate) and composites of the aforementioned materials. Synthetic biodegradable polymeric materials are often preferred for the application in tissue-engineered scaffolds because they minimize the chronic foreign body reaction and lead to the formation of completely natural tissue. That is to say, they can form a temporary scaffold for mechanical and biochemical support.

Homopolymers of lactide and its copolymers with glycolide and ε-caprolactone are the most extensively investigated synthetic biodegradable polymers used clinically. This is because their biodegradable profile can be readily controlled by changing the molecular weight and copolymer compositions. In addition, it is recognized that the constituting monomers are nontoxic because they are all metabolites in the body.

Many naturally occurring scaffolds or scaffolds made of a natural polymeric material can be used as biomaterials for tissue engineering purposes. One example is the extracellular matrix (ECM), a very complex biomaterial controlling cell function that designs natural and synthetic scaffolds to mimic specific functions.

In one embodiment, natural synthetic polymeric materials include, but are not limited to alginate, collagens (gelatine), fibrins, albumin, gluten, elastin, fibroin, hyarulonic acid, cellulose, starch, chitosan (chitin), scleroglucan, esinan, pectinic acid, galactan, curdlan, gellan, levan, emulsan, dextran, pullulan, heparin, silk, chondroitin-6-sulphate, or polyhydroxyalkanoates. Much of interest in these natural polymers comes from their biocompatibility, relative abundance and commercial availability, and ease of processing.

In another embodiment, natural synthetic polymeric materials include, but are not limited to poly(β-hydroxybutyrate), poly(malic acid), chitin, chitosan, hyaluronic acid, pectin, pectic acid, galactan, starch, dextran, pullulan, agarose, heparin, alginate, chondroitin-6-sulfate, collagen, gelatin, fibrin, albumin, gluten, polypeptide, elastin, fibroin, hydroxyapatite, calcium phosphate, tricalcium phosphate, or tetracalcium phosphate.

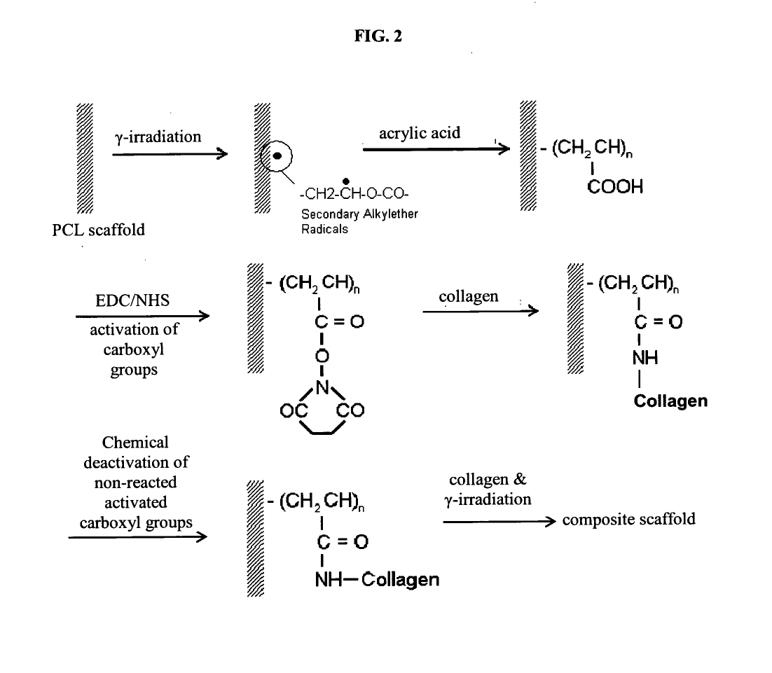

In a first step of the method of manufacturing a composite scaffold, the three-dimensional porous scaffold as described above is exposed to gamma-rays. Irradiation of the scaffold with gamma-rays creates free radicals on the surface of the scaffold. The kind of radial formed at the surface depends on the scaffold material. Examples of different radicals formed on the surface of the scaffold include, but are not limited to alkyl, allyl, polyenyl, peroxy or peroxide radicals. In one example in which a PCL scaffold is used, the radical formed at the surface of the polymeric backbone structure of the scaffold is a secondary alkylether radical as illustrated for example in FIG. 2.

Gamma irradiation of the scaffold can be carried out under air, i.e. the scaffold is placed on a carrier and is exposed to the surrounding atmosphere, for example in a gamma irradiation chamber.

The radiation dosage can be between about 10 kGy to about 30 kGy or can be at least 10 kGy to 20 kGy or can be below 30 kGy (Gy=gray; unit for absorbed radiation dose). The dosage range chosen for a specific material should not induce significant degradation of the mechanical properties of the scaffold material. However, in general radiation dosages above 30 kGy will start to dramatically degrade the scaffold material. This does not exclude that for certain materials higher dosages than 30 kGy can be used.

Using higher dosages can also reduce the time that is necessary to ensure uniform radical formation at the surface of the scaffold (time×dosage rate (10 kGy/h)). The time for the irradiation step can be between about 15 or 30 minutes to about 3 hours (h) depending on the dosage and scaffold material. Also, a higher radiation dosage also increases the number of radicals formed at the surface of the scaffold material. A person skilled in the art can easily determine the necessary time and dosage for a specific scaffold material using the methods referred to in the experimental section further below. Using gamma-rays allows the treatment of scaffolds of any size.

To prevent decay or decomposition of the radicals formed upon exposure of the scaffold to gamma rays, the exposure to gamma-rays can be carried out under vacuum or the scaffold carrier is cooled or is made of ice.

Before contacting the scaffolds which have been exposed to the gamma-rays with the solution comprising the unsaturated carboxylic acid, it is also possible to contact the scaffold or immerse the scaffold in liquid nitrogen to avoid decay of the radicals.

In the next step the scaffold which has been exposed to gamma-rays is brought in contact with an unsaturated carboxylic acid for scaffold grafting. The unsaturated carboxylic acid can be an unsaturated carboxylic acid or derivatives thereof. To increase the efficiency of the scaffold grafting, the unsaturated carboxylic acid comprises equal or less than 12 C-atoms.

As used herein, the term “carboxylic acid” also includes the polycarboxylic acids and those acid anhydrides, such as maleic anhydride, wherein the anhydride group is formed by the elimination of one molecule of water from two carboxyl groups located on the same polycarboxylic acid molecule.

In one embodiment, the unsaturated carboxylic acid is an acrylic acid or derivatives thereof. Examples of acrylic acids or derivatives thereof which can be used include, but are not limited to acrylic acid, methacrylic acid, methyl methacrylic acid, ethacrylic acid, alpha-chloroacrylic acid, alpha-cyano acrylic acid, beta methyl-acrylic acid (crotonic acid), alpha-phenyl acrylic acid, sorbic acid, alpha-chloro sorbic acid, angelic acid, cinnamic acid, p-chloro cinnamic acid, itaconic acid, citraconic acid, mesaconic acid, glutaconic acid, aconitic acid, maleic acid, fumaric acid, or tricarboxy ethylene. In one example, acrylic acid is used.

It one embodiment a distilled form of the above mentioned unsaturated carboxylic acid is used to avoid contacting the radicals with polymerization inhibitors. Commercially available unsaturated carboxylic acids mostly include such polymerization inhibitors to prevent polymerization during storage of the product.

The content of unsaturated carboxylic acid in a solution comprising the unsaturated carboxylic acid can be between about 1 wt % to about 45 wt % based on the total weight of the solution or can have a content of at least 1 wt %. In another embodiment, the content is below 45 wt % or between about 2 to about 20% based on the total weight of the solution. The reaction efficiency will be higher when using a higher concentration of unsaturated carboxylic acid which will also increase acidic conditions. A concentration of an unsaturated carboxylic acid which exceeds the above limits can affect the property of the scaffold because of the strong acidic environment. However, depending on the material it can be possible that higher contents of unsaturated carboxylic acids can be used in some cases.

The time for the reaction between the radicals at the surface of the scaffold and the unsaturated carboxylic acid depend on the concentration of the unsaturated carboxylic acid. For example, considering a content range of 1 to 45% the reaction time can be between 5 minutes (1 wt %) to about 60 minutes (45 wt %). In one example, 30 minutes were used for a 10 wt % solution of acrylic acid or 10 minutes for a 20 wt % solution of acrylic acid. Also, the longer the reaction time the more carboxyl groups are generated at the surface of the scaffold. The temperature for this reaction can be varied. Higher temperatures speed up the reaction, i.e. the formation of carboxyl groups at the surface of the scaffold. However, the temperature should be selected to be below the melting point of the material which forms the scaffold.

To avoid that oxygen which can be comprised in the solution of the unsaturated carboxylic acid inhibits the subsequent radical-initiated polymerization, oxygen content can be reduced by continuously passing an inert gas, such as argon, into the solution comprising the unsaturated carboxylic acid.

To prevent homopolymerization of the monomer during grafting or upon storage of the aqueous monomer solution (i.e. the unsaturated carboxylic acid), a suitable amount of ferrous salt or cupric salt may be added to the solution of the unsaturated carboxylic acid. Such homopolymerization inhibitor may be preferably added in an amount of from about 0.0025 wt. % to about 2 wt. % of the solution of the unsaturated carboxylic acid or from about 0.01 to about 2 wt. %, or from about 0.1 wt. % to about 1 wt. % of the solution of the unsaturated carboxylic acid.

The homopolymerization inhibitor can be a ferrous salt or a copper salt. The homopolymerization inhibitor can include, but is not limited to ammonium iron (II) sulphate or copper sulphate.

In case no homopolymerization inhibitor is used or in addition to adding a homopolymerization inhibitor, the scaffold is washed after the reaction of the free radicals at the surface of the scaffold with the unsaturated polymeric acid. Washing can be carried out in a suitable solution, such as water or distilled water. In such a washing step the residual homopolymer can be removed.

Irrespective of the washing step referred to in the previous paragraph, the grafted scaffold obtained can optionally be washed or rinsed in water for a prolonged period before starting with the activation of the carboxyl groups now uniformly distributed at the surface of the grafted scaffold. The washing or rinsing period can be between about 1 or 6 h to about 24 hours or at least 6 hours.

Before bringing the grafted scaffold in contact with the first polymer solution, the carboxyl groups at the surface of the scaffold are activated. Activation of the carboxyl groups can be carried out via chemical activation.

Methods for chemical activation of carboxyl groups are known in the art. For example, the chemical activation of the carboxyl groups can be carried out by using for example a solution of diazoalkanes, or diazoacetyl compounds, or dicyclohexylcarbodiimide (DCC), or carbonyldiimidazole, or 1-(3-dimethylaminopropyl)-3-ethylcarbondiimide, or a combination of N-hydroxysuccinimide (NHS) and 1-(3-dimethylaminopropyl)-3-ethylcarbondiimide (EDC).

For example, 1-ethyl-3-(3-dimethylaminopropyl)-carbodiimide (EDC) is a water-soluble derivative of carbodiimide. Carbodiimide catalyzes the formation of amide bonds between carboxylic acids and amines by activating carboxyl to form an O-urea derivative (see for example FIG. 2). This derivative reacts readily with nucleophiles. In general, EDC can be used to make ether links from alcohol groups and ester links from acid and alcohols or phenols, and peptide bonds from acid and amines. N-Hydroxysuccinimide (NHS) is often used to assist the carbodiimide coupling in the presence of EDC. The reaction includes formation of the intermediate active ester (the product of condensation of the carboxylic group and N-hydroxysuccinimide) that further reacts with the amine function to yield finally the amide bond.

The pH range for the activation reaction of the carboxyl groups can be selected to be between about 5 to 6 while the reaction time depends on the temperature. In one example, the reaction time was about 2 h. The activation reagent comprised in the solution referred to above can be dissolved in a suitable buffer to form the solution. An example for such a buffer is 2-(N-Morpholino)ethanesulfonic acid (MES).

After activation of the carboxyl groups the scaffold is contacted with or immersed in a first polymer solution in a first contacting step to allow reaction of the activated carboxyl groups with the polymer in the polymer solution. In this reaction a thin layer of polymer is grafted onto the surface of the scaffold. In one embodiment a natural polymeric material can be used like the natural polymeric materials referred to above.

The concentration of the polymer in the polymer solution can be between about 0.1 mg/ml to about 300 mg/ml, or at least 0.1 mg/ml, or at least 1 mg/ml, or between about 1 mg/ml to 100 mg/ml, or between about 1 to 8 mg/ml. A higher concentration of the polymer in the first polymer solution results in a faster gelation of the polymer at the surface of the scaffold, a stronger mechanical strength of the scaffold as well as a smaller pore size inside the scaffold.

The contact time is variable and depends on the polymer used. In one embodiment, the contact time can be between about 30 minutes to about 12 h or between about 30 minutes to about 3 h. The contact time depends largely on the temperature. For example, in some embodiments, a temperature of 4° C. results in a contact time of about 12 hours while at 37° C. the contact time is about 30 minutes.

After contacting the grafted scaffold with the first polymer solution, the carboxyl groups which have not reacted with the polymer in the polymer solution can be deactivated. The deactivation can be a chemical deactivation. Chemical deactivation of carboxyl groups is known in the art and can be carried out for example by contacting the scaffold with a primary amine containing compound.

Primary amine containing compounds are commercially available and can include, but are not limited to tris(hydroxymethyl)aminomethane (TRIS), lysine, glycine, hydroxylamine, methylamine, ethanolamine, ethylamine, propylamine, isopropyl amine, butylamine, sec-butylamine, iso-butylamine, hexylamines (all conformational types), heptylamines (all conformational types), octylamines (all conformational types), nonylamines (all conformational types), decylamines (all conformational types) and mixtures or combinations thereof. The pH range in solutions of primary amine containing compounds can be between about 8 to 10.

The scaffold which has been contacted with the first polymer can be subjected to the washing step after contacting of the scaffold with the first polymer or in case a deactivation of the carboxyl groups is carried out, after the deactivation of the carboxyl groups. The scaffold can be washed in a buffer, such as PBS, for at least one day or for between about 1 to 3 days or 2 days.

In the following step the scaffold now comprising a first layer of a polymer at its surface is contacted or immersed in a second polymer solution. The polymer can be a non-biodegradable polymeric material or a biodegradable polymeric material. In a further embodiment, the biodegradable polymeric material can be a natural polymeric material or a synthetic polymeric material. The polymeric materials can be the same or different from the polymeric materials which are defined above for the use as scaffold materials. Also, the polymer used for the second polymer solution can be the same or different from the polymer used for the first polymer solution.

While being contacted with the second polymer, the scaffold is subjected to gamma-rays for a second time. The dosage range for the gamma-rays can be between about 5 kGy to about 30 kGy. The dosage should be chosen to avoid degradation of the scaffold. Irradiation with gamma-rays has the effect that the polymer forms a hydrogel and is cross-linked to the first polymer layer which was formed upon reaction of the activated carboxyl groups with the first polymer. The layer of first polymer forms an anchor for the second polymer which is cross-linked to the first polymer via gamma-irradiation to obtain a better mechanical stability as it would be available when using only one gamma-irradiation step. Mechanical anchoring the second polymer to the first polymer also stabilizes the porous network structure forming in the already existing porous backbone structure of the scaffold. Mechanical anchoring further avoids that the hydrogel formed by the second polymer upon gamma-irradiation is washed out of the scaffold in case it gets in contact with liquids, such as blood, culturing medium, buffer or water.

In one embodiment, the polymer material used for the scaffold is the same material as the polymer material used for the first and/or second polymer solution. It is also possible that different polymer materials are used for the scaffold, the first polymer solution and the second polymer solution. In another embodiment, the polymer used in the first polymer solution is the same or different from the polymer used for the second polymer solution. The use of different polymer materials for the different components of the composite scaffold allows for example to determine the degradation time of the different components individually.

In a further embodiment it is possible to include a further chemical compound into the second polymer solution. Such a compound can include, but is not limited to antiproliferative/antimitotic agents including natural products, such as vinca alkaloids (e.g. vinblastine, vincristine, and vinorelbine), paclitaxel, epidipodophyllotoxins (e.g. etoposide, teniposide); antibiotics, such as actinomycin D, daunorubicin, doxorubicin and idarubicin; anthracyclines, mitoxantrone, bleomycins, plicamycin (mithramycin) and mitomycin; enzymes, such as L-asparaginase which systemically metabolizes L-asparagine and deprives cells which do not have the capacity to synthesize their own asparagine; antiproliferative/antimitotic alkylating agents, such as nitrogen mustards (such as mechlorethamine, cyclophosphamide and analogs, melphalan, chlorambucil); ethylenimines and methylmelamines, such as hexamethylmelamine and thiotepa; alkyl sulfonates-busulfan; nirosoureas (carmustine (BCNU) and analogs, streptozocin), trazenes-dacarbazinine (DTIC); antiproliferative/antimitotic antimetabolites, such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine{cladribine}); platinum coordination complexes (cisplatin, carboplatin), procarbazine, hydroxyurea, mitotane, aminoglutethimide; hormones (e.g. estrogen); anticoagulants (heparin, synthetic heparin salts and other inhibitors of thrombin); fibrinolytic agents (such as tissue plasminogen activator, streptokinase and urokinase); antiplatelet (such as aspirin, dipyridamole, ticlopidine, clopidogrel, abciximab); antimigratory; antisecretory (such as breveldin); antiinflammatory: such as adrenocortical steroids (cortisol, cortisone, fludrocortisone, prednisone, prednisolone, 6-alpha-methylprednisolone, triamcinolone, betamethasone, and dexamethasone), non-steroidal agents (such as salicylic acid derivatives e.g. aspirin); para-aminophenol derivatives (e.g. acetaminophen); indole and indene acetic acids (such as indomethacin, sulindac, and etodalac), heteroaryl acetic acids (such as tolmetin, diclofenac, and ketorolac), arylpropionic acids (such as ibuprofen and derivatives), anthranilic acids (such as mefenamic acid, and meclofenamic acid), enolic acids (such as piroxicam, tenoxicam, phenylbutazone, and oxyphenthatrazone), nabumetone, gold compounds (such as auranofin, aurothioglucose, gold sodium thiomalate); immunosuppressive (such as cyclosporine, tacrolimus (FK-506), sirolimus (rapamycin), azathioprine, mycophenolate mofetil); angiogenic such as vascular endothelial growth factor (VEGF), fibroblast growth factor (FGF); nitric oxide donors; anti-sense oligo nucleotides and combinations thereof.

In another embodiment, the chemical compound can include, but is not limited to an antibiotic, an anti-inflammatory agent, an anti-clotting factor, a hormone, a nucleic acid; a peptide, a cellular factor, a growth factor, a ligand for a cell surface receptor, an anti-proliferation agent, an anti-thrombotic agent, an antimicrobial agent, an anti-viral agent, a chemotherapeutic agent, and an anti-hypertensive agent.

In another embodiment, the anti-thrombotic drug can include, but is not limited to small organic molecules, such as clopidogrel, triflusal, or analog salicylic acid derivatives or a protein such as hirudine or thrombin. Illustrative examples of an anti-restenotic drug are sirolimus, also called rapamycin paclitaxel, and evolimus.

In this context, it is noted that the drug (therapeutically active agent) to be incorporated into one or more polymeric materials of the occlusion device can be a small organic molecule, a protein or a fragment of the protein, a peptide or a nucleic acid such as DNA or RNA. The term “small organic molecule” as used herein typically denotes an organic molecule comprising at least two carbon atoms, but preferably not more than 7 or 12 rotatable carbon bonds, having a molecular weight in the range between 100 and 2000 Dalton, or between 100 and 1000 Dalton, that optionally can include one or two metal atoms. The term “peptide” as used herein typically refers to a dipeptide or an oligopeptide with 2-about 40, 2-about 30, 2-about 20, 2-about 15, or 2-about 10 amino acid residues. The peptide may be a naturally occurring or synthetic peptide and may comprise—besides the 20 naturally occurring L-amino acids—D-amino acids, non-naturally occurring amino acids and/or amino acid analogs. With “protein” is meant any naturally occurring polypeptide that comprises more than 40 amino acid residues. The protein can be a full length protein or a truncated form, for example, an active fragment. Illustrative examples of proteins include, but are not limited to antibodies or other binding proteins with antibody like properties (for example, affibodies or lipocalin muteins knows as “Anticalins®”) for selected cell receptors, growth factors such as VEGF (Vascular Endothelial Growth Factor) and similar factors for transmitting signals, cardiovascular therapeutic proteins or cardiac hormones and active fragments thereof or prohormones or preprohormones of such cardiac hormones (these hormones or the prohormones can either be peptides as defined herein, if they have less than 40 amino acid residues of a protein, should there polypeptide sequence contain more the 40 amino acid residues). Further examples for cardiovascular therapeutic agents can be peptides or DNA such as the DNA for nitric oxide. Examples of nucleic acid molecules include sense or anti-sense DNA molecules (if expression of a target gene is to be controlled) or the coding sequence (either alone or in gene-therapy vector, for example) of a therapeutically active protein that is to be produced. In such a case, the nucleic acid may code for a protein that promotes wound healing as described in International patent application WO 97/47254, for example.

All drugs or therapeutic agents mentioned above can be used alone or in any combination thereof in the polymer material of this embodiment of the invention. If a drug is contained, the drug can be incorporated into the polymer material by admixing, impregnating, or the like, wherein the drug does not necessarily need to be uniformly distributed within the polymer material.

Inclusion of chemical compounds into the composite scaffold allows to use such a composite scaffold as drug delivery device.

In a further aspect, the present invention refers to a method of manufacturing a composite scaffold seeded with cells. This method can comprise providing a composite scaffold as referred to herein and seeding cells into the composite scaffold. The cell seeded composite scaffold can be immersed in a suitable culture medium. The composition of the culture medium depends on the cell type seeded therein. Suitable cell types include prokaryotic or eukaryotic cell types. The composite scaffold is particularly useful for anchorage dependent cells.

In a further aspect, the present invention refers to a composite scaffold which has been obtained according to the method described herein. Such a composite scaffold can be used for different purposes, such as for tissue engineering, drug delivery, cell culturing or in bio-chips, or bio-sensors, or as Bio-MEMs or for some micro-device surface modification. The method referred to herein is useful for such applications because the possibility of a uniform surface modification of the inner surface through the deep penetration property of gamma rays. Thus, opening of those small and/or integrated devices for free radical uniform induction can be avoided.

In still another aspect, the present invention refers to a porous three-dimensional composite scaffold having a compressive modulus of between about 5 to 50 MPa and having a pore size of between about 20 to 200 μm. The method referred to herein allows to produce porous three-dimensional composite scaffolds for different applications which require different compressive modulus. For example, for a trabecular bone, i.e. a composite scaffold which is supposed to replace a trabecular bone, the compressive modulus should be about 50 MPa. For a composite scaffold replacing cartilage the compressive modulus should range between about 5 to 10 MPa. In one embodiment referred to herein, the compressive modulus comprising a scaffold made of PCL has a compressive modulus of between about 6 to 7 MPa and can thus be used for the replacement of cartilage tissue.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Manufacture of a Composite Scaffold

In one example, γ-radiation was carried out in air at room temperature under 10 kGy power to produce the desired active radicals on the material surfaces throughout the entire porous PCL scaffold as illustrated in FIG. 2. The PCL scaffolds (8 mm×8 mm×6 mm, Osteopore, Singapore) were placed in ice to avoid overheating. The γ-radiated scaffolds were immediately lowered into liquid nitrogen and quickly transferred for the subsequent graft polymerisation experiment.

The γ-radiated scaffolds were quickly lowered into 50 mL of distilled acrylic acid (AAc) (10%) injected slowly with 50 mg ammonium iron (II) sulphate for 10 minutes at 30° C. Dissolved oxygen, which can inhibit the subsequent radical-initiated polymerisation, was reduced by continuously passing Argon gas into the AAc. After graft polymerisation, the scaffolds were stirred in distilled water to remove the residual homopolymer from the scaffolds' surfaces. The AAc grafted scaffolds (PCL-AAc) were rinsed in deionized water for 24 h. The carboxylic acid group on the AAc was activated with 1-(3-dimethylaminopropyl)-3-ethylcarbondiimide (EDC) and N-hydroxysuccinimide (NHS) in 2-(N-morpholino)ethanesulfonic acid buffer (MES) in a 2 h reaction. The EDC/NHS solution used was made up of 0.68 g of EDC and 0.386 g of NHS dissolved in 100 mL of MES buffer at a pH of 6. The PCL-AAc scaffolds were then lowered into 3.0 mg/mL Collagen I (PureCol™, Inamed Corporation, Fremont, Calif., USA) for overnight at 4° C. and subsequently immersed and stirred in 0.5% ethanolamine solution (chemical deactivation of non-reacted activated carboxyl groups) in 1×PBS at room temperature for 2 h to deactivate carboxyl groups.

Finally, the scaffolds were washed in 1×PBS for 2 days to remove the collagen which was absorbed physically on the surface. The pore size of the scaffold was reduced in a controlled fashion via collagen cross-linking. The collagen is further immersed in 6 mg/ml collagen and bubbles trapped within the scaffolds were removed via centrifugation. The collagen immersed scaffolds were treated with 25 kGy γ-radiation for crosslinking and sterilization. The collagen crosslinked scaffolds were then washed for 2 days in 1×PBS. The scaffolds were subsequently dried under reduced pressure in a freeze-drier for 24 h.

Uniformity of Gamma-Irradiation Grafting in Composite Scaffolds

Toluidine Blue O (TBO) Assay

Carboxyl groups on the surface of carboxylated PCL scaffolds were quantified by TBO assay as described previously, but with some modifications. PCL scaffolds were cut into blocks of 2 mm thickness with a razor blade. Cross-sections of PCL scaffolds under different modification treatments were incubated in 3 mL of TBO solution (0.5 mM in 0.1 mM NaOH, pH 10) in a 12-well culture plate for 5 h at room temperature under constant shaking. Uncomplexed dye was removed by washing with excess amount of NaOH solution (pH 10) two times (3 h each time). Complexed TBO on the surface of carboxylated PCL scaffolds was desorbed from the surface by incubating scaffold sections in 3 mL of 50% acetic acid solution for 5 h under constant shaking. TBO concentration in acetic acid solution was determined by absorbance measurement at 633 nm with a microplate reader (Safire², Tecan). Carboxyl group density on the surface was calculated from complexed TBO content, assuming that TBO complexes with the carboxyl groups on the scaffold surface at 1:1 ratio.

Acid Orange II (AO) assay

Acid Orange (AO) II assay was used for determining the degree of functionalisation, i.e., the density of primary and secondary amine groups on the surface of collagen-immobilised PCL scaffolds. Collagen-immobilised PCL scaffolds were cut into blocks of 2 mm thickness with a razor blade. Cross-sections of PCL scaffolds of different treatments were immersed in a solution of 500 μM Acid Orange II in deionised water at pH 3 at which there is a protonation of amines. After shaking overnight at room temperature, the cross-sections were washed twice with deionised water at pH 3. Amount of bound dye was quantified after detachment in deionised water at pH 12, which was achieved after 30 mins of shaking at room temperature. This was because deprotonation of amines occurred at basic pH values. AO concentration in deionised water was determined by absorbance measurement at 492 nm with a microplate reader (Safire², Tecan). Surface density of amine groups was quantified by comparison with a standard curve of known concentrations of amine groups.

Results

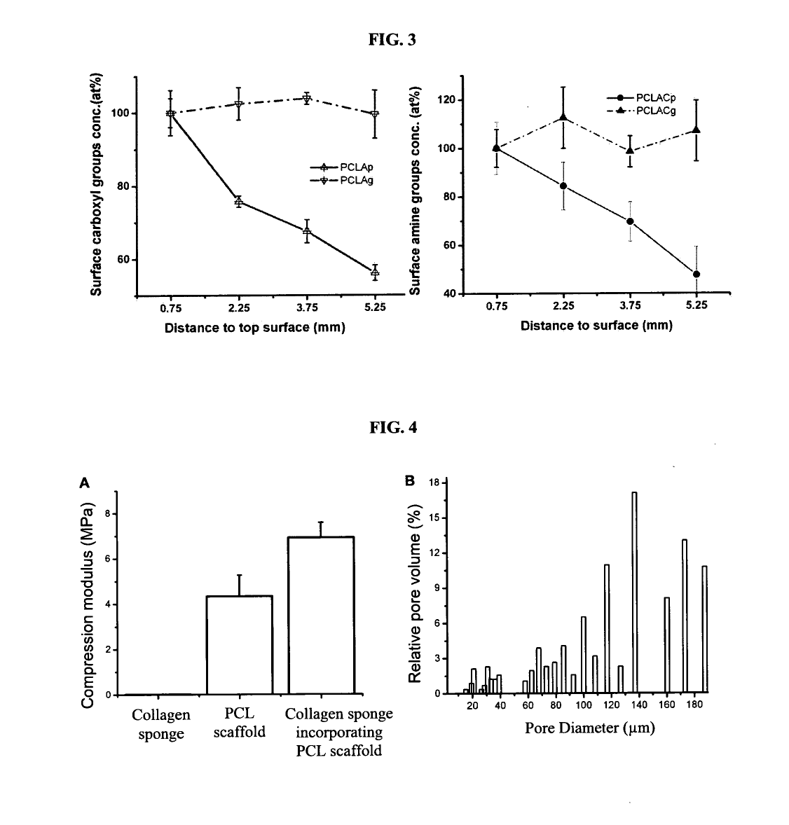

To quantitatively examine the distribution of chemical elements through the scaffold from interior to periphery during the surface grafting process, TBO and AO II assays were used to determine the surface concentrations of carboxyl and amine groups respectively. These assays were used in conjunction with mechanical sectioning of surface-modified scaffolds by gamma or plasma induction (FIGS. 3(A) and 3(B)).

TBO is a basic dye, which can form an ion complex with carboxylic group on the surface at pH 10 and which desorbs under acidic environment (50% acetic acid solution). On the contrary, AO is an acidic dye which forms an ion complex with amine group on the surface at pH 3 and which desorbs under pH 12. The surface densities of carboxyl and amino groups were obtained by analyzing the total amount of TBO and AO in the desorbed solution through colorimetry, respectively. The results showed concentration gradients for both carboxyl groups and amine groups on the surfaces of PCLA and PCLAC scaffolds whereby surface grafting was induced by plasma treatment (PCLAp and PCLACp), but not for scaffolds whereby surface grafting was induced by gamma irradiation (PCLAg and PCLACg), as seen in FIG. 3. In particular, only 56% of carboxyl groups and 47% of amine groups appeared on the bottom surfaces of PCLAp and PCLACp scaffolds when normalised against the concentrations on the top surface of respective scaffold.

Mechanical Properties of PCL-Collagen Composite Scaffolds

Porosity Measurement by Mercury Porosimeter

The internal structure of PCL-collagen scaffolds-pore size distribution, total pore area and porosity were measured by PASCAL 140 mercury porosimeter (Thermo Finnigan, Italy, S.p.A.) with S-CD6 dilatometer. Sample preparation and measurements were performed according to instructions from the manufacturer.

Compression Modulus Testing of PCL-Collagen Scaffolds

Compression tests were performed on an Instron Micro-Tester 5848 (Instron Co., Canton, Mass., U.S.A.) at a speed of 0.5 mm/min and at a temperature of 25±2° C. The compression modulus was calculated from the slope of the initial linear portion of the stress—strain curve. Five scaffolds were tested for each group.

Scanning Electron Microscope

The morphology of the scaffolds and hepatocytes in the scaffolds was characterized by scanning electron microscopy. Samples were fixed with glutaraldehyde (3% in PBS) for 30 min. The samples were dried in sequential concentrations of alcohol (70%, 90% and 100%) and post-fixed with an aqueous solution of osmium tetraoxide (OsO₄) (1%) at 4° C. for 30 min. Then, samples were freeze-dried overnight and cut with a razor blade and gold-coated with an ion sputter coater (JFC-1200, Jeol, Japan) at 15 mA for 80 s. Photomicrographs were acquired by scanning electron microscope (JSM-7400M, Jeol, Japan).

Results

It is essential that scaffold have suitable mechanical properties to hold and support the cells after seeding. The compression modulus of the scaffolds was evaluated for the collagen sponge, PCL scaffold and the PCL-collagen scaffold (FIG. 4(A)). It was found that the collagen sponge without the PCL frame has very low compression modulus and the PCL scaffold has 4 times higher compression modulus than collagen sponge. The mechanical strength of the scaffold increased significantly in comparison to PCL scaffold alone after cross-linking collagen to the PCL scaffold. Pore sizes within the PCL-collagen scaffolds were also evaluated using a porosimeter (FIG. 4(B)). About 70% of the pore volume has a pore size of between 100 μm to 180 μm and the rest of the 30% of the pore volume has a pore size of less than 100 μm.

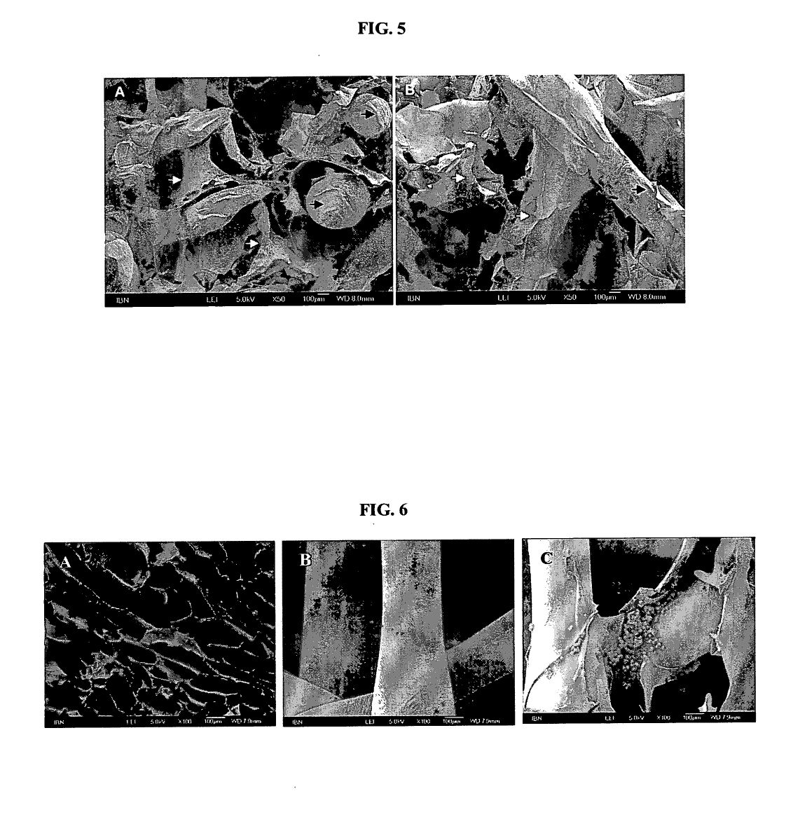

Further characterization of the PCL-collagen scaffolds was performed via visualization under scanning electron microscope to characterize the morphology of the scaffolds (FIG. 5). This is essential to evaluate the suitability of the scaffolds for cell seeding. The scanning electron microscope images show the PCL framework, covered with cross-linked collagen plates, thus, significantly increases the area for cells attachment. The scaffolds are highly porous and have uniform distribution of collagen on the top and middle. This is concurrent with the data obtained using the porosimeter.

ECM coatings such as collagen have been shown to be essential for anchorage-dependent cells attachment and maintenance of cell number. The PCL-collagen scaffold used in this example is suitable for the culture of anchorage-dependent cells due to its high surface area, suitable ECM environment and also small pore size to allow optimal cells attachment and entrapment. The biocompatibility and suitability to be used as a tissue engineering scaffolds of this composite scaffold is demonstrated in the following example.

Cellular Compatibility of PCL-Collagen Composite Scaffolds

Hepatocytes Culture

Hepatocytes were cultured for the required amount of time in Williams' E medium supplemented with 10 mmol/L nicotinamide (Sigma-Aldrich), 0.2 mmol/L ascorbic acid 2-phosphate (Sigma-Aldrich), 20 ng/mL epidermal growth factor, 20 mmol HEPES (Gibco), 0.5 μg/mL insulin, 0.1 μmol/L dexamethasone, 100 U/mL penicillin G, and 100 μg/mL streptomycin. Hepatocytes were seeded into the scaffolds at a cell density of 1.8×10⁶ cells/scaffold (as determined using a hemacytometer). Scaffolds with hepatocytes were cultured in 12-well plates in a cell culture incubator (37° C., 95% humidity and 5% CO₂).

Cell Morphology in Scaffold

The morphology of hepatocytes in PCL-collagen scaffold was characterized by SEM. Samples were fixed with glutaraldehyde (3% in PBS) for 30 min at room temperature. After repeated rinsing in PBS, constructs were further fixed with an aqueous solution of osmium tetraoxide (OsO₄) (1%) at 4° C. for 30 min at room temperature. After washing with deionised water and dehydration through a graded ethanol series, the scaffolds were dried by hexamethyldisilazane. Dried scaffolds were cut into 2 mm thickness with a razor blade and gold-coated with an ion sputter coater at 15 mA for 60 s. SEM micrographs were acquired with a field emission scanning electron microscope.

Cell Attachment and Metabolism Activity of Hepatocytes in Scaffold

The number of hepatocytes attached to the scaffold after 24 h of cell culture was evaluated by quantifying the DNA content of a crude cellular homogenate of the hepatocytes using PicoGreen dsDNA Quantitation Kit (Molecular Probes). The metabolism activity of hepatocytes cultured in different scaffolds was examined using the colorimetric AlamarBlue™ assay (BioSource International Inc., Camarillo, USA).

Results

Surface chemistry of scaffolds (cell substrates) plays a pivotal role in altering cell attachment and metabolism in tissue engineering applications and biological responses elicited in these scaffolds are especially important as they reveal the clues that pave the way for tissue regeneration.

Hepatocyte as anchorage-dependent cell that is highly sensitive to environmental cues were seeded in the hybrid PCL-collagen scaffold. Thereafter, cell seeding, cell attachment and metabolism in this scaffold were examined. Table 1 shows the cell seeding efficiencies in collagen sponge, PCL and PCL-collagen scaffolds. Cell seeding efficiency was determined according to the equation below:

Seeding efficiency(%)=100(Nₒ−Nᵣ)/Nₒ

where Nₒ is the number of cells seeded in the scaffold, Nᵣ is the number of cells which remained at the bottom of 24-well plate after 2 h of cell seeding (determined by a hemacytometer).

TABLE 1
Seeding efficiencies of hepatocytes in different scaffolds

| | Collagen sponge | PCL scaffold | PCL-collagen scaffold |
|---|---|---|---|
| Seeding | 61% | 6% | 97% |
| efficiency | | | |

It was found that the efficiency of hepatocytes seeded in the PCL-collagen scaffold (composite scaffold) was the highest compared to those in collagen sponge or PCL scaffold alone. A possible explanation for this result is rendered scaffold-by-scaffold as follows:

For collagen sponge, deformation of its geometry in cell seeding process—as a result of its poor mechanical property—resulted in hepatocytes leaking from the cell seeding medium, thereby causing low cell seeding efficiency.

For PCL scaffold, hepatocytes in the cell seeding medium leaked out through interspaces between the microfibres due to poor cellular affinity of PCL and large pore size of preformed PCL scaffold, thereby resulting in low cell seeding efficiency in PCL scaffold.

For PCL-collagen scaffold (composite scaffold), microstructures formed by combination of collagen strips and aligned PCL microfibres contributed to retaining hepatocytes as well as good cell attachment on the surface of collagen strips. This implied that the PCL-collagen scaffold possessed the advantages of both PCL scaffold and collagen sponge, thereby increasing the cell loading capacity of this hybrid scaffold.

FIG. 6 shows the morphologies of hepatocytes at 24 h after cell seeding in collagen sponge (FIG. 6(A)), PCL scaffold (FIG. 6(B)) and PCL-collagen scaffold (FIG. 6(C)) examined by SEM. As predicted above, few hepatocytes were attached on the surface of PCL scaffold (FIG. 6(B)) and were present in singles or in pairs. Some hepatocytes also attached on the surface of collagen strips in collagen sponge in singles or in pairs (FIG. 6(A)). In contrast, many hepatocytes attached on the surface of collagen strips or PCL microfibers in PCL-collagen scaffold as cell aggregates (FIG. 6(C)), which was consistent with the results of cell seeding efficiencies study with the same explanation.

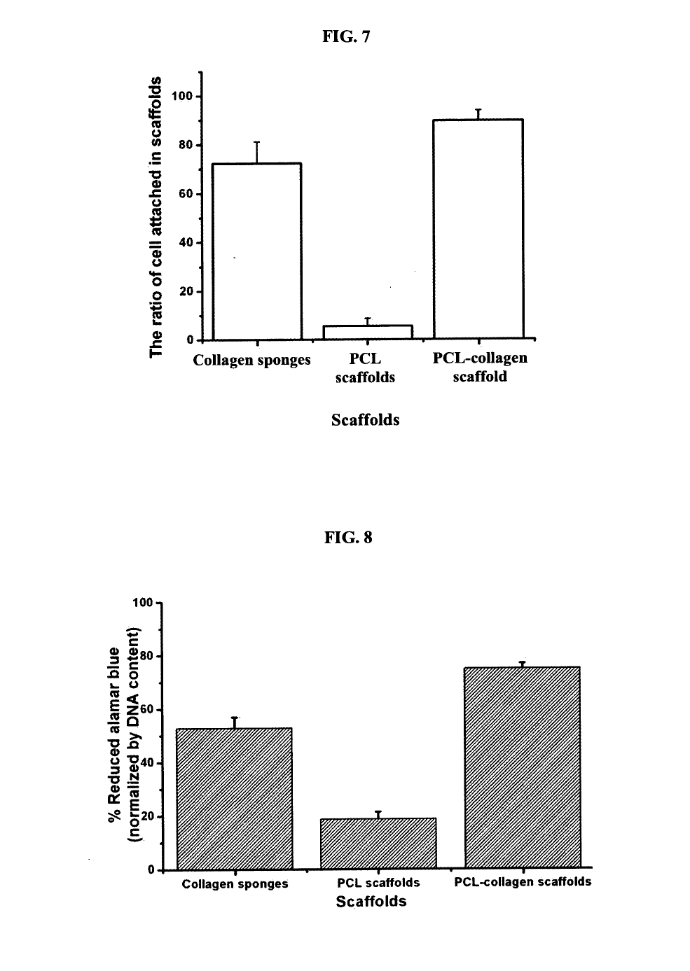

The number of hepatocytes attached in each scaffold at 24 h after cell seeding was evaluated in terms of DNA content by means of PicoGreen dsDNA Quantitation Kit. FIG. 7 shows the number of hepatocytes attached to each scaffold. Hepatocyte number in PCL-collagen scaffold was the highest probably due to: (1) Higher cell seeding efficiency in PCL-collagen scaffold than in collagen sponge and PCL scaffold; (2) RGD (arginine-glycine-aspartate) groups on the surface of collagen-grafted PCL microfibres promoted cell attachment.

The metabolic activity of hepatocytes cultured in each scaffold at 24 h after cell seeding was evaluated by AlamarBlue™ assay. FIG. 8 shows the reduction of AlamarBlue by hepatocytes cultured in collagen sponge, PCL scaffold and PCL-collagen scaffold. All data were normalised by the number of hepatocytes cultured in each scaffold. The results indicated that the reduction of AlamarBlue by hepatocytes cultured in the PCL-collagen scaffold was much higher than in collagen sponge or PCL scaffold. This result could be attributed to: (1) Biocompatible surface of PCL-collagen scaffold promoted the metabolism activity of cell, and (2) The best mass transfer among 3 kinds of scaffolds, which benefited from interconnective channels microstructure and appropriate mechanical strength, resulted in the best cell metabolism activity.

Determining the Compressive Young's Modulus

To determine the compressive modulus the following method is used. The final composite scaffold obtained with the method referred to herein were cut into blocks (5 mm length, 5 mm width and 6 mm thickness). Compression tests were done at room temperature on an Instron Micro Tester 5848 apparatus (Instron Corp., Canton, Mass., USA) using a load cell of 1 KN at a speed of 0.5 mm/min and at a temperature of 25±2° C. Compression modulus was calculated from the slope of the initial linear portion of stress-strain curve.

1. A method of manufacturing a composite scaffold comprising:
irradiating a three dimensional porous scaffold with gamma-rays;
contacting the scaffold in a solution comprising an unsaturated carboxylic acid for introducing carboxyl groups at the surface of the scaffold;
activating the carboxyl groups to obtain activated carboxyl groups;
contacting the scaffold comprising the activated carboxyl groups with a first polymer solution in a first contacting step to allow reaction of the activated carboxyl groups with the polymer in the polymer solution;
contacting the scaffold in a second contacting step with a second polymer solution; and
irradiating the scaffold which is in contact with the second polymer solution with gamma-rays to obtain the composite scaffold.

2. The method of claim 1, wherein the scaffold is made of a non-biodegradable polymeric material or biodegradable polymeric material.

3. The method of claim 2, wherein the non-biodegradable polymeric material is selected from the group consisting of polystyrene, polyvinylalcohol (PVA), polyhydroxyethyl-methacrylate (pHEMA) and poly(N-isopropylacrylamide) (PNIPAAm).

4. The method of claim 2, wherein the biodegradable material is a synthetic polymeric material or a natural polymeric material.

5. The method of claim 4, wherein the synthetic polymeric material is selected from the group consisting of polylactides, polyglycolides, lactide/glycolide copolymers, polycaprolactone, polyp-dioxane), poly(β-malic acid), poly(anhydrides), poly(ortho esters), polycarbonates, poly(phosphazenes), poly(amino acids), poly(phosphoric ester-urethanes), poly(cyanoacrylates), polyethylene, polyurethane, poly(butyl acrylate), poly(methyl methacrylate), poly(ethylene terephthalate) and composites of the aforementioned materials.

6. The method of claim 4, wherein the natural polymeric material is selected from the group consisting of poly(β-hydroxybutyrate), poly(malic acid), chitin, chitosan, hyaluronic acid, pectin, pectic acid, galactan, starch, dextran, pullulan, agarose, heparin, alginate, chondroitin-6-sulfate, collagen, gelatin, fibrin, albumin, gluten, polypeptide, elastin, fibroin, hydroxyapatite, calcium phosphate, tricalcium phosphate, and tetracalcium phosphate.

7. The method according to claim 1, wherein the gamma-ray dosage for irradiation of the three dimensional porous scaffold is between about 10 kGy to about 30 kGy.

8. The method of claim 1, wherein the three dimensional porous scaffold is cooled or kept under vacuum during the irradiating step with gamma-rays.

9. The method of claim 1, wherein the unsaturated carboxylic acid comprises 12 or less than 12 carbon atoms.

10. The method of claim 9, wherein the unsaturated carboxylic acid is an acrylic acid or derivatives thereof.

11. The method of claim 10, wherein the acrylic acid or the derivative thereof is selected from the group consisting of acrylic acid, methacrylic acid, methyl methacrylic acid, ethacrylic acid, alpha-chloroacrylic acid, alpha-cyano acrylic acid, beta methyl-acrylic acid (crotonic acid), alpha-phenyl acrylic acid, sorbic acid, alpha-chloro sorbic acid, angelic acid, cinnamic acid, p-chloro cinnamic acid, itaconic acid, citraconic acid, mesaconic acid, glutaconic acid, aconitic acid, maleic acid, fumaric acid, and tricarboxy ethylene.

12. The method of claim 1, wherein the content of unsaturated carboxylic acid in the solution is between about 1 wt % to about 45 wt % based on the total weight of the solution.

13. The method of claim 12, wherein the content of unsaturated carboxylic acid in the solution is between about 2 wt % to about 20 wt % based on the total weight of the solution.

14. The method of claim 1, wherein the solution comprising an unsaturated carboxylic acid is aerated with an inert gas.

15. The method of claim 1, wherein a homopolymerization inhibitor is added to the solution comprising an unsaturated carboxylic acid.

16. The method of claim 15, wherein the homopolymerization inhibitor is of a ferrous salt or a cupric salt.

17. The method of claim 16, wherein the homopolymerization inhibitor is selected from the group consisting of ammonium iron (II) sulphate and copper sulphate.

18. The method of claim 1, wherein the chemical activation of the carboxyl groups is carried out by using a solution comprising diazoalkanes, or diazoacetyl compounds, or carbonyldiimidazole, or dicyclohexylcarbodiimide (DCC), or 1-(3-dimethylaminopropyl)-3-ethylcarbondiimide or N-hydroxysuccinimide/1-(3-dimethylaminopropyl)-3-ethylcarbondiimide.

19. The method of claim 1, wherein the polymer in the first polymer solution is a natural polymeric material.

20. The method of claim 19, wherein the natural polymeric material is selected from the group consisting of poly(β-hydroxybutyrate), poly(malic acid), chitin, chitosan, cellulose, hyaluronic acid, pectin, pectic acid, galactan, starch, dextran, pullulan, agarose, heparin, alginate, chondroitin-6-sulfate, collagen, gelatin, fibrin, albumin, gluten, polypeptide, elastin, fibroin, hydroxyapatite, calcium phosphate, tricalcium phosphate, and tetracalcium phosphate.

21. The method of claim 1, wherein the concentration of the polymer in the first polymer solution is between about 0.1 to 300 mg/ml.

22. The method of claim 1, wherein the scaffold is contacted with the first polymer solution for a time period between about 30 minutes to about 3 hours.

23. The method of claim 1, wherein the method further comprises after the step of contacting the scaffold with a first polymer solution the step of deactivating activated carboxyl groups which have not reacted with the polymer in the polymer solution;

24. The method of claim 23, wherein the chemical deactivation of the carboxyl groups which have not reacted with the polymer in the polymer solution is carried out by using a primary amine containing compound.

25. The method of claim 24, wherein the primary amine containing compound is selected from the group consisting of tris(hydroxymethyl)aminomethane, lysine, glycine, hydroxylamine, methylamine, ethanolamine, ethylamine, propylamine, isopropylamine, butylamine, sec-butylamine, iso-butylamine, hexylamines, heptylamines, octylamines, nonylamines, decylamines and mixtures or combinations thereof.

26. The method of claim 1, wherein the polymer in the second polymer solution is a biodegradable or non-biodegradable polymeric material.

27. The method of claim 26, wherein the non-biodegradable polymeric material is selected from the group consisting of polystyrene, polyvinylalcohol (PVA), polyhydroxyethyl-methacrylate (pHEMA) and poly(N-isopropylacrylamide) (PNIPAAm).

28. The method of claim 26, wherein the biodegradable material is a synthetic polymeric material or a natural polymeric material.

29. The method of claim 28, wherein the synthetic polymeric material is selected from the group consisting of polylactide, polyglycolide, lactide/glycolide copolymer, polycaprolactone, poly(p-dioxane), poly(β-malic acid), poly(anhydrides), poly(ortho esters), polycarbonates, poly(phosphazenes), poly(amino acids), poly(phosphoric ester-urethanes), poly(cyanoacrylates), polyethylene, polyurethane, poly(butyl acrylate), poly(methyl methacrylate), poly(ethylene terephthalate), polyvinyl alcohol and composites of the aforementioned materials.

30. The method of claim 28, wherein the natural polymeric material is selected from the group consisting of poly(β-hydroxybutyrate), poly(malic acid), chitin, chitosan, hyaluronic acid, pectin, pectic acid, galactan, starch, dextran, pullulan, agarose, heparin, alginate, chondroitin-6-sulfate, collagen, gelatin, fibrin, albumin, gluten, polypeptide, elastin, fibroin, hydroxyapatite, calcium phosphate, tricalcium phosphate, and tetracalcium phosphate.

31. The method of claim 1, wherein the gamma-ray dosage for irradiation of the scaffold which is in contact with the second polymer solution is between about 5 kGy to about 30 kGy.

32. The method of claim 1, wherein the second polymer solution further comprises a chemical compound.

33. The method of claim 32, wherein the chemical compound is selected from the group consisting of an antiproliferative/antimitotic agent, an enzyme, an antiproliferative/antimitotic alkylating agent, a platinum coordination complex, a hormone, an anticoagulants, a fibrinolytic agent, an antiplatelet compound, an antimigratory compound, an antisecretory compound, an anti-inflammatory compound, a para-aminophenol derivative, an heteroaryl acetic acid, an arylpropionic acid, an anthranilic acid, an enolic acid, a nabumetone, a gold compound, an immunosuppressive compound, an angiogenic compound, a nitric oxide donor, an anti-sense oligo nucleotide and combinations thereof.

34. The method of claim 32, wherein the chemical compound is selected from the group consisting of an antibiotic, an anti-inflammatory agent, an anti-clotting factor, a hormone, a nucleic acid, a peptide, a cellular factor, a growth factor, a ligand for a cell surface receptor, an anti-proliferation agent, an anti-thrombotic agent, an antimicrobial agent, an anti-viral agent, a chemotherapeutic agent, and an anti-hypertensive agent.

35. A method of manufacturing a composite scaffold seeded with cells, comprising:
providing a composite scaffold as referred to in claim 1; and
seeding cells into the composite scaffold.

36. The method of claim 35, wherein the cells are prokaryotic or eukaryotic cells.

37. The method of claim 36, wherein the eukaryotic cells are anchorage dependent eukaryotic cells.

38. (canceled)

39. (canceled)

40. (canceled)

41. (canceled)
